(19) United States
(12) Patent Application Publication
(10) Pub. No.: US 2007/0172912 A1
(43) Pub. Date: Jul. 26, 2007

(54) Method for fluorescently staining tissue

(75) Inventors: Akira Yamamoto, Tokyo (JP); Yusuke Iimori, Tokyo (JP); Mizue Saze, Tokyo (JP); Mariko Ishiguro, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/656,373

(22) Filed: Jan. 23, 2007

(30) Foreign Application Priority Data
Jan. 25, 2006 (JP) 2006-16678

(51) Int. Cl. G01N 1/30; G01N 33/48

(52) U.S. Cl. 435/40.5

(57) ABSTRACT

It is intended to provide a method for fluorescently staining a tissue accurately and clearly by convenient procedures and a staining agent used in this method. The present invention provides a method for fluorescently staining a tissue, comprising treating a tissue with a solution comprising fluorescein or a salt thereof and then fluorescently observing the treated portion under an acidic condition of lower than pH 7.

### BACKGROUND OF THE INVENTION

1. Field of the Invention

The present invention relates to method for fluorescently staining a living body tissue or a living body-derived tissue conveniently and clearly and to a staining agent used in this method.

2. Description of the Related Art

The acquisition of cross-sectional images of living body tissues is of extreme importance in the medicobiological field. Cross-sectional images of tissues extracted from living bodies could previously be obtained by chemical fixation, dehydration, slicing, and staining.

The development of a confocal imaging system and the prevalence of a confocal laser scanning microscope enabled noninvasive observation of cells and tissues in the observation of sections of biological samples with intricately multi-layered cells and connective tissues. Medical endoscopes equipped with a built-in confocal imaging system have recently been developed.

The observation of fluorescent images in a confocal optical system involves procedures for staining a living body tissue and a sample derived therefrom with a fluorescent dye. When a living body tissue is observed with a confocal scanning microscope and a medical endoscope equipped with this microscope, a reagent for staining is required to be biologically safe.

Fluorescein has conventionally been used as a fluorescent contrast agent safe to living bodies in funduscopy and so on, wherein an aqueous solution thereof is intravenously injected (Non-Patent Document 1). Alternatively, a tissue can be stained by directly sprinkling a reagent for staining onto the tissue surface, without passing through blood vessels such as veins. The method involving directly sprinkling a staining solution onto tissue surface is effective means for staining extracted organs. In in-vivo tissue staining as well, this method, as compared with the staining method involving intravenous injection and perfusion, has the advantage of being able to reduce the influence of a staining solution on living bodies in that the method requires only a small amount of the staining solution and has no dye perfusion to a site which does not require perfusion in the living bodies.

The fluorescein solution sprinkled onto the living body tissue surface is permeated into the internal region of the tissue, and a part of the solution is left as a liquid pool on the tissue surface. If the tissue is irradiated with excitation light in this state, both the permeated fluorescein molecule and the fluorescein molecule remaining on the tissue surface emit fluorescence, making the distinction between the stained and unstained sites blurry. Therefore, in the method involving sprinkling the staining agent onto the tissue surface, a free fluorescein molecule remaining on the unstained site had to be removed by washing.

The free dye can be removed by washing the stained tissue sample several times with an appropriate buffer or solution. However, the sample to be observed has a quite delicate surface and must be washed with care in avoiding the deformation or damage of the sample and the denaturation of the tissue. Thus, the washing procedures must be performed gently and carefully. Moreover, excessive washing also causes the detachment of the dye (decoloring) from the stained tissue.

On the other hand, when an extracted tissue sample is stained without performing chemical fixation and observed with a confocal scanning microscope, the autolysis of the tissue as well as cell or protein denaturation occurs during washing procedures, presenting the problem of the sample having a state different from that within the living body.

### [Non-patent Document 1] Gastroenterology, 127 (3), 706-713, 2004

An object of the present invention is to provide a method for fluorescently staining a tissue accurately and clearly by convenient procedures and a staining agent used in this method.

### SUMMARY OF THE INVENTION

Thus, the present inventors have focused attention on fluorescein and conducted various studies. Fluorescein dissolved in an aqueous solution exhibits fluorescence at alkaline pH and hardly exhibits fluorescence in an acidic solution. Thus, fluorescein has conventionally been used in an alkaline solution form and fluorescent observation has been made under an alkaline condition. However, the present inventors have found out from various studies that, totally unexpectedly, fluorescein bound with a tissue exhibits clear fluorescence under an acidic condition in spite of the fact that fluorescein hardly exhibits fluorescence in an acidic solution. Accordingly, if a tissue treated with fluorescein is placed under an acidic condition during fluorescent observation, only fluorescein bound with the tissue is selectively capable of fluorescent staining, while fluorescein unbound with the tissue hardly exhibits fluorescence. Consequently, the present inventors have found out that a clear and accurate fluorescently stained image of a tissue can be obtained without performing complicated washing procedures. Based on these findings, the present invention has been completed.

Specifically, the present invention provides a method for fluorescently staining a tissue, comprising treating a tissue with a solution comprising fluorescein or a salt thereof and then fluorescently observing the treated portion under an acidic condition of lower than pH 7.

The present invention also provides a fluorescent staining agent for a tissue comprising a solution comprising fluorescein or a salt thereof, which is intended for treating a tissue and then fluorescently observing the treated portion under an acidic condition of lower than pH 7.

The present invention further provides use of a solution comprising fluorescein or a salt thereof for the production of a fluorescent staining agent for a tissue intended for treating a tissue and then fluorescently observing the treated portion under an acidic condition of lower than pH 7.

According to the staining method of the present invention, a clear and accurate stained image can be obtained because only fluorescein bound with a tissue is selectively capable of fluorescent staining. Namely, fluorescein is highly dependent on pH in general, and its fluorescent property tends to be enhanced more at higher pH values. Only a fluorescein molecule that is permeated into the internal region of a living body tissue and bound with the tissue emits strong fluorescence by staining the living body tissue under a previously unimaginable condition wherein a fluorescent observation condition is set to an acidic condition. Under this condition, a free fluorescein molecule does not emit fluorescence. Therefore, a fluorescently stained image of the living body tissue can be observed without performing complicated and invasive procedures such as washing after staining and perfusion.

### BRIEF DESCRIPTION OF THE DRAWINGS

FIG. 1 is a diagram showing the pH dependence of fluorescence emission of fluorescein;

FIG. 2 is a diagram showing a fluorescently stained image of the large intestine treated with a fluorescein solution with pH 4.0 and then observed fluorescently; and

FIG. 3 is a diagram showing a fluorescently stained image of the large intestine treated with a fluorescein solution with pH 9.0 and then observed fluorescently.

### DETAILED DESCRIPTION OF THE PREFERRED EMBODIMENTS

In a method of the present invention, a tissue is treated with a solution comprising fluorescein or a salt thereof. In this context, the tissue encompasses both a living body tissue and a living body-derived tissue (extracted tissue). The living body tissue includes the esophagus, stomach, duodenum, small intestine, large intestine, rectum, oral cavity, and lumens of urinary organs. The living body-derived tissue includes tissues derived from these living body tissues and further includes tissues derived from a variety of organs and muscular tissues.

The fluorescein or the salt thereof used in the present invention includes fluorescein, fluorescein sodium, and fluorescein potassium, and fluorescein and fluorescein sodium salts are particularly preferable. Of them, fluorescein and a fluorescein disodium salt (uranine) are particularly preferable. The fluorescein has green fluorescence (excited at 490 nm) around 520 nm and is therefore well excited with Ar laser light (488 nm, 514 nm). An aqueous solution of uranine is an acidic dye intramolecularly having a xanthene skeleton, which, albeit in small amounts, emits green fluorescence on exposure to light. The absorption maximum of the uranine dye is 450 to 490 nm, though differing depending on solvents.

Preferably, means for treating the tissue in the present invention is application of the solution comprising fluorescein or a salt thereof to the tissue or immersion of the tissue into the solution. In this context, application is preferable in a case a living body tissue is used as the tissue to be treated. Application means includes spraying, sprinkling and so on. When a living body-derived tissue is used as the tissue, application or immersion is used. The amount of the solution applied may be an amount that permits the solution to spread throughout the tissue to be treated.

The treated portion has only to be placed under an acidic condition of lower than pH 7 during fluorescent observation, and thus the solution used may be a basic solution with not lower than pH 7 or an acidic solution with lower than pH 7. When a basic solution with not lower than pH 7 comprising fluorescein or a salt thereof is used, the tissue is treated with the solution, and the treated portion may then fluorescently be observed after washing with an acidic solution with lower than pH 7. Alternatively, when an acidic solution with lower than pH 7 comprising fluorescein or a salt thereof is used, the tissue is treated with the solution and may directly be observed fluorescently.

The solution with not lower than pH 7 comprising fluorescein or a salt thereof is prepared by adding a basic substance, for example, sodium hydroxide, sodium acetate, sodium hydrogen phosphate, or glycine, which adjusts aqueous solution pH to not lower than 7, to an aqueous solution comprising fluorescein or a salt thereof. Preferable pH is 7 to 10, particularly preferably 7 to 8. Preferably, the pH is adjusted with a buffer, more preferably with, for example, sodium phosphate, tris(hydroxymethyl)aminomethane hydrochloride, lysine hydrochloride, or arginine hydrochloride.

The solution with lower than pH 7 comprising fluorescein or a salt thereof is prepared by adding an acidic substance, for example, phosphoric acid, hydrochloric acid, carbonic acid, an inorganic acid, or a nontoxic organic acid (e.g., acetic acid or citric acid), which adjusts aqueous solution pH to lower than 7, to an aqueous solution comprising fluorescein or a salt thereof. Preferable pH is 4 to 6.5, particularly preferably 6 to less than 7. Preferably, the pH is adjusted with a buffer, more preferably with, for example, a nontoxic acidic buffer solution such as sodium phosphate, sodium acetate, sodium citrate, or sodium carbonate.

A fluorescein concentration in the solution comprising fluorescein or a salt thereof is preferably 0.001 mg/mL to 50 mg/mL, more preferably 0.1 to 10 mg/mL. Fluorescein with a concentration not lower than 10 mg/mL is in a solution form that is easily deposited at not higher than pH 5. Fluorescein with a concentration not higher than 0.001 mg/mL has a low staining property.

As described above, the treated portion of the tissue to be stained is placed under an acidic condition of lower than pH 7 by washing with an acidic solution or treatment with an acidic solution with lower than pH 7 comprising fluorescein or a salt thereof. Therefore, free fluorescein does not emit fluorescence, while only fluorescein bound with the tissue emits fluorescence.

The fluorescent observation may be measured by irradiation with excitation light. Preferably, the fluorescently stained image is observed with a fluorescence microscope, a fluorescence endoscope, or a confocal imaging system. The confocal imaging system includes a confocal scanning microscope and an endoscope with a confocal imaging system.

A fluorescent staining agent of the present invention comprises only a solution comprising fluorescein or a salt thereof when the solution is an acidic solution with lower than pH 7. On the other hand, the fluorescent staining agent comprises a solution comprising fluorescein or a salt thereof in combination with an acidic solution with lower than pH 7 (for washing) when the solution is a basic solution with not lower than pH 7.

### EXAMPLES

Next, the present invention will be described more specifically with reference to Examples.

### Example 1

pH-Dependent Fluorescence Emission of Fluorescein

The pH of an aqueous solution of fluorescein sodium (1 mg/mL) was adjusted with 0.1 M sodium phosphate buffer solution, followed by measurement at an excitation wavelength of 490 nm and a fluorescence wavelength of 530 nm.

A microplate reader (manufactured by Corona, MTP-800AFC) was used in the fluorescent measurement.

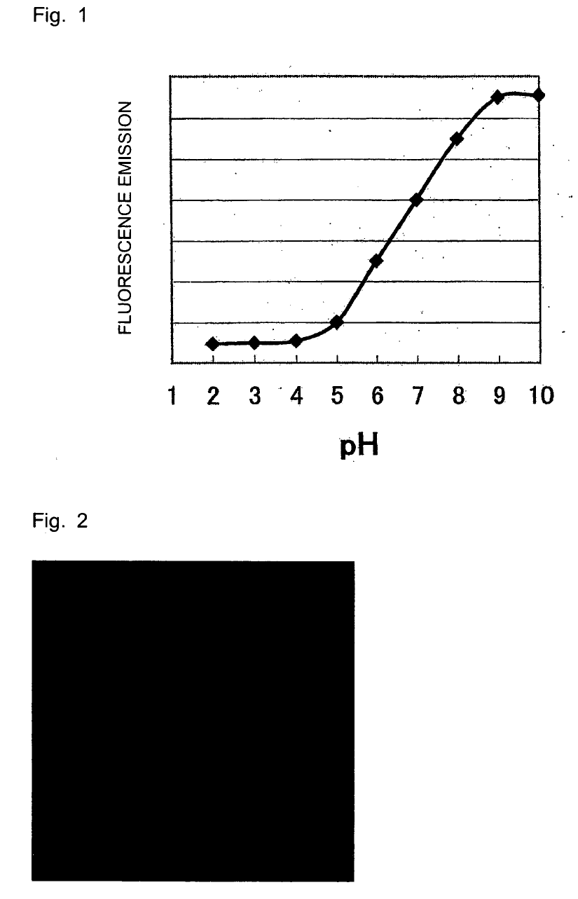

As seen from the result shown in FIG. 1, the fluorescence intensity of fluorescein gets stronger at higher pH values.

### Example 2

Fluorescein Sodium Staining Test on Rat Large Intestine

The formalin-fixed large intestines of rats (8-week-old, male) were used to observe a difference in staining property against pH. The large intestines were washed for 10 seconds with a phosphate buffered saline (137 mmol/L NaCl, 8.1 mmol/L Na₂HPO₄, 2.7 mmol/L KCl, 1.57 mmol/L KH₂PO₄, hereinafter referred to as PBS (−)) and then immersed into solutions of fluorescein sodium (manufactured by Sigma, F6377, hereinafter referred to as F—Na) adjusted and diluted to 1 mg/mL with acidic, alkaline, and neutral buffer solutions. The neutral, alkaline, and acidic buffer solutions used were 0.1 M sodium phosphate buffer solution (pH 7.0), 0.1 M borate buffer solution (pH 9.1), and 0.4 M NaH₂PO₄ buffer solution (pH 4.65), respectively. After further washing for 10 seconds with PBS (−), the resulting large intestines were fixed with 10% formalin-acid buffer solution and observed with a confocal scanning microscope (manufactured by Leica Microsystems, TCS-SP2).

Although all the tissue samples were well stained, F—Na diluted with the buffer solutions with pH 7.0 and 9.0 gave an increased background and was therefore unsuitable for cell observation.

### Example 3

Difference in Permeation and Fluorescent Property of Dye Depending on Changes in Solvent pH

The large intestines of rats (8-week-old, male) were extracted and subjected to a staining test.

F—Na (manufactured by Sigma, F6377) was adjusted to 1 mg/mL and diluted with 0.1 M sodium phosphate buffer solution (pH 7.0), 0.1 M borate buffer solution (pH 9.1), and 0.4 M NaH₂PO₄ buffer solution (pH 4.65) to prepare their respective 0.1 mg/mL solutions, into which the tissues were then immersed for 1 minute. After further washing for 10 seconds with PBS (−), the resulting tissues were observed with a confocal scanning microscope (manufactured by Leica Microsystems, TCS-SP2). The results are shown in Table 1.

TABLE 1

| | | Fluorescence intensity | | |
|---|---|---|---|---|
| | | examined visually | | |
| | | (indicated in five tiers) | | |
| | | Free | Fluorescein | State of observed |
| | | fluorescein | in internal | image (clarity |
| | | in | region of | of fluorescent |
| Solvent | pH | liquid pool | tissue | image) |
| 0.4 M NaH₂PO₄ | 4.65 | 1 | 3 | Clear |
| solution | | | | |
| 0.1 M Na | 7.0 | 5 | 3 | Not clear but |
| phosphate | | | | tissue is |
| buffer | | | | identifiable |
| solution | | | | |
| 0.1 M Na | 9.1 | 4 | 3 | Liquid pool has |
| borate | | | | stronger |
| buffer | | | | fluorescence |
| solution | | | | |

### Example 4

Tissue staining observation was performed in the pH region of 4 to 7 under an isotonic condition of F—Na (manufactured by Sigma, F6377).

The large intestines of rats (8-week-old, male) were extracted. They were washed with a phosphate buffered saline (137 mmol/L NaCl, 8.1 mmol/L Na₂HPO₄, 2.7 mmol/L KCl, 1.5 mmol/L KH₂PO₄, 4.4 mol/L CaCl₂.2H₂O, 1.6 mmol/L MgCl₂.6H₂O, hereinafter referred to as PBS (+)) and then stained by immersion for 1 minute into F—Na (0.1 mg/mL) adjusted to each pH. The stained large intestines were observed with a confocal scanning microscope (manufactured by Leica Microsystems, TCS-SP2).

Here, a control test using a saline was conducted at the same time. This is because a saline is used as a solvent for F—Na used in intravenous injection and so on.

Imaging conditions for the confocal scanning microscope included a confocal pinhole diameter of 1.00 airy, and two types of lenses, 20× and 63× oil-immersion lenses, were used. The microscope was programmed to automatically correct a gain value, and the observation was performed at the optimum luminance.

Fluorescein Na/D.W. was adjusted to 5 mg/mL and diluted to 0.1 mg/mL with a citric acid-phosphoric acid buffer solution with each pH and a saline. The large intestines were immersed into the adjusted staining solutions, then washed with PBS (+), and observed. The results are shown in Table 2.

TABLE 2

| | Fluorescence intensity | | |
|---|---|---|---|
| | examined visually | | |
| | (indicated in five tiers) | | |
| Citric | | Fluorescein in | State of observed |
| acid-phosphoric | Free | internal | image |
| acid buffer | fluorescein in | region of | (clarity of |
| solution pH | liquid pool | tissue | fluorescent image) |
| 4.0 | 1 | 3 | Clear |
| 5.0 | 1 | 4 | Clear |
| 6.0 | 2 | 4 | Clear |
| 7.0 | 3 | 4 | Not clear but tissue |
| | | | is identifiable |
| Saline | 3 | 4 | Liquid pool |
| | | | has stronger |
| | | | fluorescence |

### Example 5

Slices of the large intestines used in Example 4 were observed, and the degree of permeation of a staining solution was observed. Fluorescein sodium was adjusted to 0.1 mg/mL in the same way as in Example 4 by dilution with solutions with pH 4.0, 5.0, 6.0, and 7.0 and a saline.

Imaging was performed with a fluorescence microscope (manufactured by ZEISS, LSM510) under imaging conditions including a pinhole diameter of 1.0 and Max, and 20× and 40× lenses were used.

The observation of the slices showed that a portion on the order of 50 μm in the epithelial cell is well stained, and strong fluorescence is observed in the portion.

In the observation using the staining solutions with varying pH values, no significant difference was observed in the degree of permeation into the tissues.

As is evident from Examples 1 to 5, fluorescein in both free and tissue-bound forms emits fluorescence under an alkaline condition, while only the tissue-bound form emits fluorescence under an acidic condition.

### Example 6

Observation was performed at varying concentrations at constant pH set to 6.0.

The concentration of F—Na (manufactured by Sigma, F6377) was set to 10, 1.0, 0.1, 0.01, or 0.001 mg/mL. After adjustment to each concentration with a citric acid-phosphoric acid buffer solution (pH 6.0), observation was performed with a confocal scanning microscope (manufactured by Leica Microsystems, TCS-SP2).

Imaging conditions for the confocal scanning microscope included a confocal pinhole diameter of 1.00 airy, and two types of lenses, 20× and 63× oil-immersion lenses, were used. The microscope was programmed to automatically correct a gain value, and the observation was performed at the optimum luminance.

Although the stained image was confirmed with the low F—Na concentrations, namely at 0.01 and 0.001 mg/mL, a clear image could not be obtained due to a low luminance.

As a result, a clear image exhibiting tissue permeability was obtained in the region of 0.1 to 10 mg/mL.

### Example 7

Slices of the large intestines used in Example 6 were observed, and the degree of permeation of a staining solution was observed.

The concentration of fluorescein sodium was set to 100, 10, 1.0, 0.1, 0.01, or 0.001 mg/mL in the same way as in Example 6. After adjustment to each concentration with a citric acid-phosphoric acid buffer solution (pH 6.0), imaging was performed with a confocal scanning microscope (manufactured by ZEISS, LSM510) under imaging conditions including a confocal pinhole diameter of 1.0 and Max, and 20× and 40× lenses were used.

As a result, in the observation with varying fluorescein sodium concentrations, the staining solutions with higher concentrations stained the lamina propria mucosae more darkly.

TABLE 3

| | Lamina | | | |
|---|---|---|---|---|
| Concentration | propria | Epithelial | | |
| (mg/mL) | mucosae | cell | Nucleus | Goblet |
| 10 | +++ | +++ | − | − |
| 1.0 | ++ | +++ | − | − |
| 0.1 | ++ | +++ | − | − |
| 0.01 | + | +++ | − | − |
| 0.001 | + | +++ | − | − |

+: The larger the number of + becomes, the more favorably the tissue is stained
−: Unstained

### Example 8

Observation of Staining Property of Fluorescein Sodium with Fluorescence Microscope

After fluorescein sodium (hereinafter, referred to as F—Na) staining, observation was performed with a fluorescence microscope (manufactured by Leica Microsystems, DM IRB).

Formalin-fixed rabbit small intestines were used as samples and observed under conditions of pH 4.0 and 9.0.

F—Na was adjusted to 1 mg/mL with a saline and readjusted to 0.01 mg/mL by dilution with 0.1 M phosphoric acid buffer with each pH.

The staining solution adjusted to each pH was applied to the formalin-fixed rabbit small intestines without performing washing procedures. The excessive staining solution was removed by adsorption with a filter paper, followed by observation with a fluorescence microscope. An objective lens used had a magnification of 40 times.

As a result, the staining solution with pH 4.0 stained the rabbit small intestinal villi even without performing washing procedures after staining and offered a clear stained image. However, when observation was conducted directly after staining with the staining solution with pH 9.0, fluorescence was emitted from a liquid outside the tissue rather than from the villi, and an unclear image was obtained. The 0.1 M phosphoric acid buffer solution (pH 9.0) used in the dilution required washing procedures for removing the neighboring fluorescent solution.

### Example 9

Staining by Intestinal Infusion of Fluorescein Sodium

In living bodies, the digestive organs constantly secrete mucus, whereas secreted mucus is uptaken into the bodies and digested. To verify the advantages of the present invention under an environment similar to the use in an endoscope, the staining property of fluorescein sodium for the large intestine of a living body was observed.

The staining of the large intestine of a living body at two pH values of acidic and alkaline regions as well as observation with a confocal scanning microscope (manufactured by Leica Microsystems, TCS-SP2) was performed.

A mouse (9-week-old, male) was injected under anesthesia with 500 μL of F—Na (pH 4, 1 mg/mL) from the upper portion of the large intestine by use of a syringe (Terumo 27G, 0.4 mm). After 10 minutes, the large intestine was extracted, and the interior of the intestine was washed with PBS (+).

The extracted site of the large intestine was obtained by excising 5.5 to 6.5 cm from the caecum.

As seen from FIG. 2 (gain value of 480 V, approximately 0 μm from the epithelium), the tissue structure was observed clearly by using F—Na with pH 4.0, and the goblet cell was unstained.

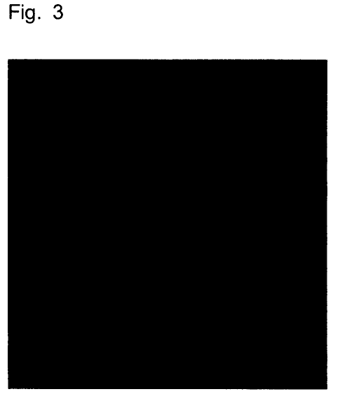

Furthermore, a staining solution of F—Na with pH 9.0 (1 mg/mL, 500 μL) was used to perform observation. The staining solution with pH 9.0 provided little difference in fluorescent luminance between the stained site and a liquid pool containing free F—Na and gave an unclear tissue image, as in the previous observation made with respect to different pH values (FIG. 3).

### Example 10

Difference in Staining Property Depending on Washing Solution pH in Fluorescein Sodium Staining

Fluorescein sodium (hereinafter, referred to as F—Na) emits strong fluorescence in an aqueous solution with not lower than pH 7. In the staining of tissue surface with an aqueous solution of F—Na with not lower than pH 7, both F—Na molecules present in the internal region of the tissue and in a liquid pool on the tissue surface exhibit a fluorescent property and therefore make the stained tissue observation difficult.

F—Na present in the liquid pool can be removed by washing the tissue surface. However, F—Na in the internal region of the tissue is also eluted due to the washing procedures, resulting in reduced staining effect.

Thus, after the staining of a tissue with an aqueous solution of F—Na with not lower than pH 7, a small amount of an acidic solution with lower than pH 7 was applied (sprinkled) to the tissue to thereby delete the fluorescent property of free F—Na that did not participate in staining. Then, observation after staining was performed. The results are shown in Table 4.

TABLE 4

| | | | |
|---|---|---|---|
| Staining | pH 9.0 → | pH 7.0 → | pH 4.0 → |
| solution pH → | pH 4.0 | pH 4.0 | pH 7.0 |
| washing | | | |
| solution pH | | | |
| Internal | Stained and | Stained and | Stained and |
| region of | fluorescence is | fluorescence is | fluorescence is |
| tissue | exhibited | exhibited | exhibited |
| Liquid pool | No fluorescence | No fluorescence | No fluorescence |
| portion | | | |
| Lamina | Stained and | Stained and | Stained but image is |
| propria | fluorescence is | fluorescence is | unclear |
| mucosae | exhibited | exhibited | |
| Visibility | Outlines of cells in | Outlines of cells in | Cell shape is |
| of cell shape | epithelial tissue | epithelia tissue | identifiable but |
| | can be identified | can be identified | contrast is low |
| Visibility | Crypt is clearly | Crypt is clearly | Crypt cannot be |
| of crypt | observed because of | observed because of | identified due to |
| shape | strong contrast | strong contrast | fluorescence |
| | between liquid pool | between liquid pool | emitted from liquid pool |
| | and internal region | and internal region of tissue | |
| | of tissue | | |

What is claimed is:

1. A method for fluorescently staining a tissue, comprising treating a tissue with a solution comprising fluorescein or a salt thereof and then fluorescently observing the treated portion under an acidic condition of lower than pH 7.

2. The method for fluorescently staining a tissue according to claim 1, wherein the solution comprising fluorescein or a salt thereof is an acidic solution with lower than pH 7.

3. The method for fluorescently staining a tissue according to claim 1, wherein the solution comprising fluorescein or a salt thereof is a basic solution with not lower than pH 7, and the treated portion is fluorescently observed after washing with an acidic solution with lower than pH 7.

4. The method for fluorescently staining a tissue according to claim 1, wherein the tissue is a living body tissue or a living body-derived tissue.

5. The method for fluorescently staining a tissue according to claim 1, wherein the treatment of the tissue with the solution comprising fluorescein or a salt thereof is application of the solution to the tissue or immersion of the tissue into the solution.

6. The method for fluorescently staining a tissue according to claim 1, wherein the fluorescent observation is observation with a fluorescence microscope, a fluorescence endoscope, or a confocal imaging system.

7. A fluorescent staining agent for a tissue comprising a solution comprising fluorescein or a salt thereof, which is intended for treating a tissue and then fluorescently observing the treated portion under an acidic condition of lower than pH 7.

8. The fluorescent staining agent for a tissue according to claim 7, wherein the solution comprising fluorescein or a salt thereof is an acidic solution with lower than pH 7.

9. The fluorescent staining agent for a tissue according to claim 7, wherein the solution comprising fluorescein or a salt thereof is a basic solution with not lower than pH 7, and the treated portion is fluorescently observed after washing with an acidic solution with lower than pH 7.

10. The fluorescent staining agent for a tissue according to claim 7, wherein the tissue is a living body tissue or a living body-derived tissue.

11. The fluorescent staining agent for a tissue according to claim 7, wherein the treatment of the tissue with the solution comprising fluorescein or a salt thereof is application of the solution to the tissue or immersion of the tissue into the solution.

12. The fluorescent staining agent for a tissue according to claim 7, wherein the fluorescent observation is observation with a fluorescence microscope, a fluorescence endoscope, or a confocal imaging system.
